(19) United States
(12) Patent Application Publication
(10) Pub. No.: US 2023/0218305 A1
(43) Pub. Date: Jul. 13, 2023

(54) Head Tourniquet

(71) Applicant: Ashley Schubert, Bowling Green, KY (US)

(72) Inventor: Ashley Schubert, Bowling Green, KY (US)

(21) Appl. No.: 18/095,022

(22) Filed: Jan. 10, 2023

Related U.S. Application Data
(60) Provisional application No. 63/298,289, filed on Jan. 11, 2022.

(51) Int. Cl. A61B 17/135

(52) U.S. Cl. CPC A61B 17/135; A61B 2017/00893

(57) ABSTRACT

The tournicap is the only product of its kind that has the capability of simultaneously applying pressure to the bleeding site on the head, providing heat to the wound, and preventing third factor-acidosis that would otherwise be associated with hemorrhaging. This unprecedented product is uniquely designed with a tournicap made of a white impregnated kaolin gauze that works hand-in-hand with a boniquet made of plastic possessing inflatable air bladders for infection prevention. Moreover, the tournicap comes in multiple sizes to accommodate all patients and is incredibly easy to apply and secure onto the head to ensure immediate relief.

### CROSS REFERENCE

The present non-provisional patent application claims priority to the provisional patent application 63298289 in its entirety, titled Head Tourniquet filed Jan. 11, 2021 by Ashley Schubert.

### BACKGROUND

Although the most common way to stop bleeding is by applying pressure through the use of gauze wrappings, when placed on the head, the wrapping often slips, and therefore is not properly secured. One of the other complications associated with head trauma is heat loss and current treatments that are available for dealing with such injuries do not offer any source of heat nor do they offer any remedies for infection prevention. There have been no products available as original equipment or as an aftermarket to address this problem.

The present non-provisional patent application claims priority to the provisional patent application

An apparatus to simplify managing blood loss and warmth from the head. There have been no products available as original equipment or as an aftermarket to address this problem either.

There exists a need for an apparatus such as the head tourniquet that is not being met by any known or disclosed device or system of present.

### SUMMARY OF THE INVENTION

The main purpose of the head tourniquet is to provide users with a sock cap style hemostatic elasticized gauze hat that provides both warmth and slight compression to stop the head from bleeding.

A tourniquet for a patient, the tourniquet comprising a tournicap comprising an open end and a closed end and an elastization and a pressure configured anularly inward from the elastization to the patient, a girth and a length and a plurality of tournicap bands. The disclosure further includes a roll comprising the plurality of tournicap bands rolled inward to decrease the girth and to increase the pressure to the patient proportional to the number n of tourniquet bands rolled up in the roll. The disclosure yet includes an indicator disposed on one of the plurality of tournicap bands and configured to indicate a time span the pressure is applied to the patient from the tournicap.

### BRIEF DESCRIPTION OF THE DRAWINGS

FIG. 1 is a front top perspective view of the head tourniquet in accordance with an embodiment of the present disclosure.

FIG. 2 is a manual pump bonniquet which inflates like a blood pressure cuff in accordance with an embodiment of the present disclosure.

FIG. 3 is a front perspective view of the head tourniquet with mullet sides in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

The term ‘tournicap’ is used throughout the disclosure to refer to a tourniquet is the shape of a cap for a patient. Used synonymously with the term tournicap, the term ‘bonnequet’ is used to refer to a bonnet in the shape of a tourniquet for a patient.

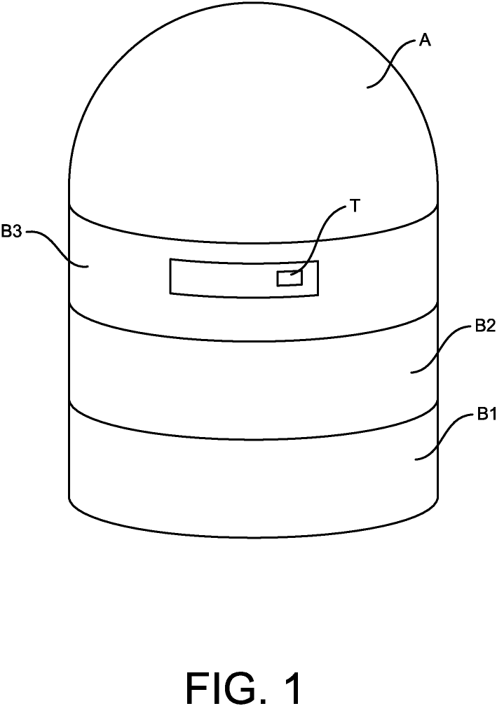

FIG. 1 is a front top perspective view of the head tourniquet showing: tournicap made of white impregnated kaolin gauze referenced as A, the bands B1, B2 and B3 which rolls up inside to resize an inside girth of the cap, or tournicap, and an application time or time span of applied pressure referenced as T, in accordance with an embodiment of the present disclosure.

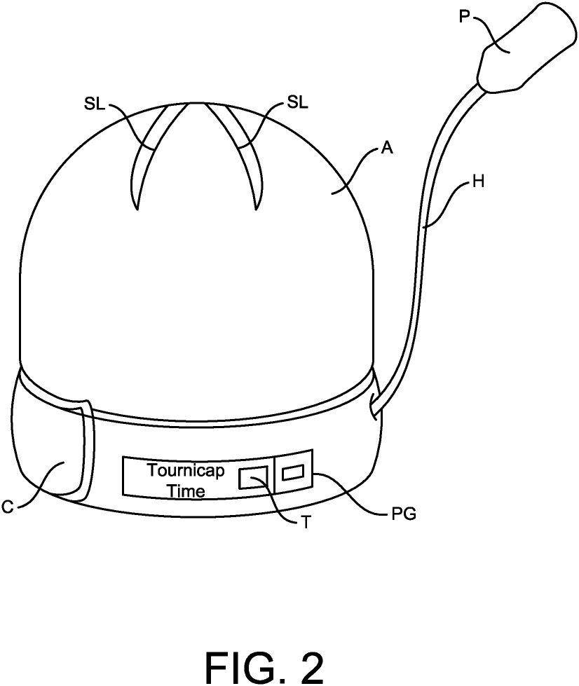

FIG. 2 is a manual pump bonniquet which inflates like a blood pressure cuff in accordance with an embodiment of the present disclosure. clotting agent powder like Celox or, even more ideally, a topical form of tranexamic acid is infused into a fabric or plastic of the tournicap, cap, bonnet and bonnequet. These items are included as a kit if a manufacturer as appropriate for markets and designs. This provides an exceptional form of bleeding control of the head/scalp that doesn’t require manual pressure. A manual pump P provides air pressure via the hose H to the cuff C. The bonniquet to inflates like a blood pressure cuff and is monitored via the pressure gauge PG. The scored lines or stretch lines SL provide opening the closed end for a tourniquet application to a leg, and arm or the patient’s head with access to the scalp.

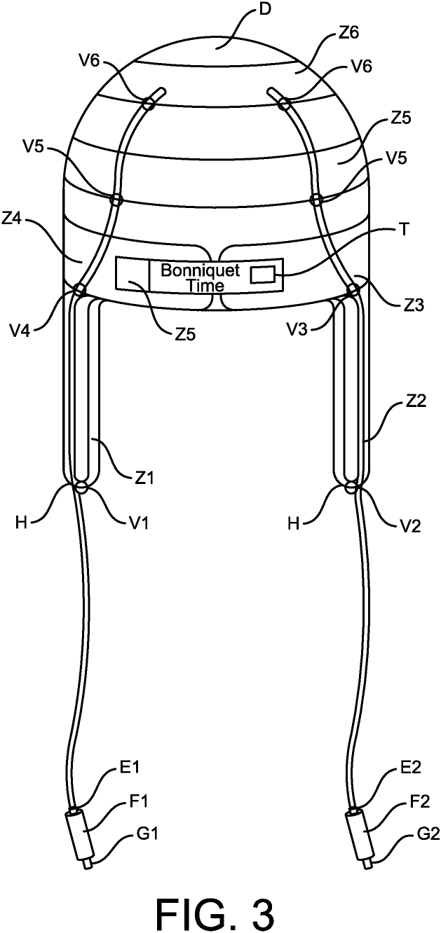

FIG. 3 is a front perspective view of the head tourniquet including mullet sides showing: bonniquet made of plastic with inflatable air bladders referenced as Z1, Z2, Z3, Z4, Z5 and Z6 where Z1 and Z2 are elongate and Z3 and Z4 are semi circular, and Z5 and Z6 are annular. The luer lock port referenced as E1 and E2, syringe ports F1 and F2 and air injection ports G1 and G2, air injection port to apply pressure, and mullet sides H in accordance with an embodiment of the present disclosure. Valves V1, V2, V3, V4, V5 and V6 allow separate control of the respective bladders in zones. Two each, one for each side, luer lock ports have separate bladders and gauges to indicate the pressures in the bladders so that it’s noted when a predetermined maximum pressure is reached. This safety feature ensures the patient the cap bladders aren’t over inflated. The applied pressure time span is indicated by T. The top of the tournicap or bonnequet D is made of plastic or a stiff material.

The present disclosed head tourniquet, also known as “Tournicap” offers a modern accessory that helps to eliminate two components from the trauma triad of death, hypothermia and coagulopathy, and therefore allows for a more effective and safe treatment. Expanding on the initial design of a tight-fitting beanie cap, the tournicap introduces a novel full head/scalp covering that also extends onto the forehead and down to the nape of the neck, if necessary. By covering the head, the cap prevents loss of heat, moreover, this cap applies a gentle pressure to the scalp and is strategically lined with a clotting agent that activates when applied to the bleeding on the scalp. To further enhance functionality, the tournicap utilizes a cutting-edge antimicrobial technology to prevent infection in lacerations and/or open skull fractures and therefore ensures patients are not placed at a higher risk for sever post-incident complications. This innovative, top-quality product ensures a highly effective treatment for those who are undergoing uncontrolled hemorrhage of the head by providing warmth to the trauma patient, controlling consistent bleeding, and combating infection prevention, in real-time. Furthermore, the tournicap may prove to be essential in the medical/therapeutic industry.

The disclosure is also used for the extremities especially in cases of amputations. Embodiments of the disclosure fit a stump of a limb to control bleeding as well. It is used furthermore in conjunction with the sprinkle clotting powders like celox powder contained inside the disclosed tourniquet.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A tourniquet for a patient, the tourniquet comprising:
a tournicap comprising an open end and a closed end and an elastization and a pressure configured anularly inward from the elastization to the patient, a girth and a length and a plurality of tournicap bands;
a roll comprising the plurality of tournicap bands rolled inward to decrease the girth and to increase the pressure to the patient proportional to the number n of tourniquet bands rolled up in the roll; and
an indicator disposed on one of the plurality of tournicap bands and configured to indicate a time span the pressure is applied to the patient from the tournicap.

2. The tourniquet of claim 1, wherein the roll also decreases a length of the elasticized cap proportional to the number n of tourniquet bands rolled up in the roll.

3. The tourniquet claim 1, wherein the tournicap comprises white impregnated kaolin gauze.

4. The tourniquet claim 1, wherein the tournicap comprises a blood anticlogging agent.

5. The tourniquet of claim 1, further comprising a plurality of inflatable air bladders in a fabric of the tournicap configured to increase the pressure applied to the patient.

6. The tourniquet of claim 1, further comprising a hook and a loop cuff configured to further increase the pressure applied to the patient via a tightening of the cuff.

7. The tourniquet of claim 1, further comprising a hose and a hand pump configured to inflate an air bladder in the tournicap and increase the pressure applied to the patient.

8. The tourniquet of claim 1, further comprising a plurality of annular inflatable air bladders in the roll configured to increase the pressure applied to the patient.

9. The tourniquet of claim 1, further comprising a hose and a hand pump configured to inflate an air bladder in the roll and increase the pressure applied to the patient.

10. The tourniquet of claim 1, further comprising a luer lock port and drug delivery hose therefrom to the tournicap.

11. The tourniquet of claim 1, further comprising a plurality of pressure zones and a control valve for each of the plurality of pressure zones.

12. The tourniquet of claim 1, further comprising a pressure gauge disposed on one of the plurality of tournicap bands and configured to indicate the pressure applied to the patient from the tournicap.

13. The tourniquet of claim 1, wherein the tournicap bands comprise a number n configured adjacent each other along the length.

14. The tourniquet of claim 1, wherein the closed end further comprises a plurality of scored lines configured to open the closed end under an axial pressure there through.

15. The tourniquet of claim 1, wherein the closed end further comprises a plurality of stretch lines configured to convert the tournicap into a tourniquet in response to an axial pressure there through.
